(19) United States
(12) Patent Application Publication
(10) Pub. No.: US 2025/0000495 A1
(43) Pub. Date: Jan. 2, 2025

(54) SYRINGE DRIVING SYSTEMS

(71) Applicant: FUNDACIÓ HOSPITAL UNIVERSITARI VALL D'HEBRON - INSTITUT DE RECERCA, BARCELONA (ES)

(72) Inventors: XAVIER SERRES CRÉIXAMS, SANT CUGAT DEL VALLÈS (ES); JORGE MENÉNDEZ FONTAL, CERDANYOLA DEL VALLÈS (ES)

(21) Appl. No.: 18/709,227

(22) PCT Filed: Nov. 14, 2022
(86) PCT No.: PCT/EP2022/081825

§ 371 Date: May 10, 2024

(30) Foreign Application Priority Data
Nov. 15, 2021 (EP) 21383034.2

(51) Int. Cl. A61B 10/02; A61M 5/20; A61M 5/31

(52) U.S. Cl. CPC A61B 10/0283; A61M 5/20; A61M 5/31; A61M 2202/206; A61M 2205/332; A61M 2205/3365

(57) ABSTRACT

A syringe driving system which may be associated to a hypodermic syringe to perform a needle biopsy and/or an injection. The system includes a remote actuator operatively connected to a handheld body through a Bowden cable, wherein the handheld body includes: a core and a cover to shield the core, the cover includes a distal opening to receive a barrel of a syringe; wherein the core has a movable portion to secure a plunger of the syringe and a fixed portion. An actuator for a syringe driving system is also provided.

This application claims the benefit of European Patent Application EP21383034 filed on Nov. 15, 2021.

The present disclosure relates to syringe driving systems and more specifically to hypodermic syringe driving systems. The systems may be related to delivering a fluid to a subcutaneous tissue of a subject and/or aspirating a fluid sample from a subcutaneous tissue of a subject.

### BACKGROUND

Needle biopsies, also described as fine needle aspiration biopsies (FNAB), are procedures comprising the aspiration of specific tissues samples, such as those tissues suspicious of being tumors, in which it is required to preserve the characteristics of surrounding tissues, e.g., tissue integrity, thus allowing close laboratory examination, such as histologic and cytologic examinations.

Medical practitioners that perform a needle biopsy, such as surgeons or intervention radiologists, are assumed to determine the right aspiration to be applied to a syringe in order to extract the intended amount of sample from the tissue of a subject. Moreover, the medical practitioner in some circumstances may be assumed to inject a drug to the region of interest of the subject, subcutaneously. The medical practitioner applies a pressure to a syringe commeasured to inject specific substances of particular reduced quantity and in precise locations. Examples of such substances are oncolytic viruses to be injected intratumorally. Oncolytic viruses are thought to cause direct destruction of cancer cells and to stimulate immune system responses to fight hosting the cancer. The oncolytic viruses may be engineered for tumor selectivity. Other procedures take advantage of alcohol injections such as in the case of ultrasound-guided ethanol injection which has been used with promising results in the treatment of papillary thyroid cancer patients with limited number of lymph-node metastases. The injection procedure depends on the pressure applied by the medical practitioner. Particularly, a variation of pressure may alter the speed and the amount of medical drug injected to the subject. Therefore, there is a risk of lack of accuracy and repeatability of the procedure.

On the one hand, the ultrasound-guided injections procedure may allow determining the location of interest with high accuracy. On the other hand, a drawback of the current systems of injection is the scant precision in controlling the speed of injection and the amount of substance to be infused subcutaneously due to the manual, non-controlled holding of the injection tool, especially if the medical practitioner needs to hold multiple tools simultaneously.

Both kinds of the above procedures are done by a medical practitioner who is submitted to receive specific training. A frequent scenario involves the medical practitioner who uses one hand to hold a scanner device to allow a vision of the needle and its surroundings, while the other hand carries the syringe. It is understandable, that both procedures require high precision and extensive training in holding two different devices simultaneously. Furthermore, the medical practitioner needs to control his own force applied to the syringe and check constantly the positioning of the needle in order to reduce the risk of failure of the operation. Therefore, carrying out a process of biopsy or delicate injection by using one hand dedicated to hold the scanning probe and the other hand to perform the suction or injection results complicated and requires extensive training. Additionally, applying a pressure to the syringe or controlling the force needed for the aspiration introduce further risks of displacing the syringe and consequently losing the target tissue.

It is an object of the present disclosure to provide examples of syringe driving systems that avoid or at least reduce the above-mentioned drawbacks.

### SUMMARY

In a first aspect of the present disclosure, there is provided a hypodermic syringe driving system comprising a remote actuator operatively connected to a handheld body through a Bowden cable, wherein the handheld body comprises a core and a cover to shield the core at least partially, the cover comprises a distal opening to receive a barrel of a syringe and a proximal opening to receive the Bowden cable, wherein the core has a movable portion to secure a plunger of the syringe and a fixed portion, and wherein the Bowden cable has a sheath and a wire, the wire being displaceable with respect to the sheath, the movable and the fixed portion of the core being associated respectively with one of the wire and the sheath in such a way that the movable portion and the fixed portion are displaceable relative to each other along a longitudinal axis of the handheld body.

The systems of the present disclosure may be used along with an image guided system to improve the control of the operative scenario. The practitioner may hold both the handheld body with one hand and the image guided system with the other hand. The present system may allow reducing the effort of a medical practitioner in actuating a hypodermic syringe through the system and may facilitate the medical practitioner in handling the handheld body in combination with other tools, e.g., the image guided system. Thus, the practitioner may operate both the syringe driving system and the other device at substantially the same time or simultaneously.

The wire may be displaceable with respect to the sheath along the length of the Bowden cable.

The systems of the present disclosure comprise a handheld body and a remote actuator configured to move a Bowden cable that may be operatively connected at the one end to the remote actuator and at the other end to the handheld body. The handheld body may comprise a core comprising a movable portion configured to be associated to a plunger of the syringe, the movable portion configured to slidingly move along a longitudinal axis of the handheld body between the fixed portion of the core and the distal opening of the cover.

The implementation of a handheld body and a remote actuator may be advisable to assist the procedure of injection. Similarly, the handheld body and the remote actuator may increase the performance of a needle biopsy due to the fact that the remote actuator may allow the execution of an injection or an aspiration. The medical practitioner may also focus in holding other tools such as an ultrasound guiding probe to reach the exact location of interest while holding the handheld body and executing an injection or an aspiration.

In the context of the present disclosure the proximal opening of the cover may be understood as the opening in the portion of the cover closer to the sheath of the Bowden cable coupled to the remote actuator and the distal opening may be intended to be the opening in the most distant portion of the cover from the sheath of the Bowden cable. In some systems of the present disclosure, the distal opening and the proximal opening may be located along the longitudinal axis of the handheld body. Thus, the distal opening and the proximal opening may be located at opposite ends of the handheld body relative to the longitudinal axis.

The distal opening of the cover of the handheld body may be configured to be detachably coupled to the barrel of the syringe and the movable portion of the core may be configured to be detachably coupled to the plunger of the syringe. The fixed portion may be detachably coupled to the cover. This way, a force generated from the remote actuator may be transferred through the Bowden cable.

In some examples of the systems of the present disclosure the actuator may comprise a confirmation switch in electrical or data communication with the control unit to operate the motor. Some examples of the systems of the present disclosure may comprise a confirmation switch configured to allow an execution of a programmed sequence. Some examples of the confirmation switch of the systems of the present disclosure may comprise a pedal. In some examples, the systems of the present disclosure may comprise a pedal that may be used by the user e.g. a medical practitioner to confirm the sequence of steps defined in the control unit for controlling the force, the direction and/or the speed to be applied to the plunger of the syringe associated to the handheld body of the syringe actuator.

The presence of a pedal may allow the medical practitioner to handle the handheld body and a guidance probe without providing any extra challenges due to controlling the force and the speed of the plunger of the syringe. In some examples of the systems of the present disclosure, the confirmation switch configured to confirm the execution of the steps of the motor may help to reduce the movements to be executed by the hand of the medical practitioner and furthermore may decrease the undesired forces applied to the syringe which otherwise might move the hand in charge of holding the syringe. In an example, the system may comprise two confirmation pedals, each pedal configured to confirm the steps concerning a particular direction of movement of the motor. One pedal may concern a first direction of the movable portion and the other pedal may concern an opposite direction to the first direction. The presence of two confirmation pedals may enhance the security by reducing the risk of executing steps in undesired direction or executing steps before they can be performed.

In some examples, the core may comprise a spring-loaded wedge rotatably attached to the movable portion of the core, the wedge being able to move from a retracted status to allow a relative displacement between the movable portion and the fixed portion of the core, to an expanded status biased by a wedge spring to block the relative displacement between the movable portion and the fixed portion. Some examples may comprise a plurality of compression brake springs attached to the movable portion of the core, wherein the compression brake springs may be configured to move from a retracted status wherein the movable portion of the core may slide along the fixed portion of the core, and to an expanded status, which may constrain the compression brake springs to wedge against the inner wall of the fixed portion of the core to block the relative displacement between the movable portion and the fixed portion of the core. Some examples of the system may comprise a plurality of compression brake springs configured to wedge against the inner wall of the cover. The spring-loaded wedge may act as a protection mechanism configured to block the relative displacement of the movable portion of the core in the case wherein an excessive force is applied to the movable portion of the core and the movable portion moves relative to the main spring. Non exhaustive examples of excessive forces applied to the movable portion of the core may comprise a breakage of the wire.

In a further aspect of the present disclosure, there is provided an actuator for a hypodermic syringe driving system according to any of the examples of the systems herein presented, wherein the actuator has a control unit comprising a receiving module, a determining module, and an activating module. Examples of the control unit may comprise a receiving module to receive operational data, a determining module to determine working parameters of the motor based on the received operational data and an activating module to activate the motor based on the determined working parameters. Some examples of the working parameters may comprise the duration required to keep active a step defined in the control unit. Some examples of the working parameters in the present disclosure may comprise the initial speed required to run the motor or the operating speed of the movable portion of the core of the handheld body. Some examples of the working parameters may also comprise the direction of rotation of the motor.

In some examples, the systems of the present disclosure may be associated to a hypodermic syringe to perform a needle biopsy and/or an injection. The system may allow operating the syringe with high precision, in a controlled way. Therefore, the biopsy or injection may be performed with high precision and in a controlled way. Thus, thanks to the systems described in the present disclosure, injections and/or biopsies may be performed in areas or tissues involving significant precision such as the treatment of papillary thyroid cancer patients with limited number of lymph-node metastases. Human error in syringe actuation may be avoided. Unwanted forces on the syringe may be avoided.

The image guided system or any guidance device for guiding an injection or biopsy procedure and the syringe do not form part of the present disclosure.

### BRIEF DESCRIPTION OF THE DRAWINGS

Non-limiting examples of the present disclosure will be described in the following, with reference to the appended drawings, in which:

FIG. 1 schematically illustrates a syringe driving system according to an example;

FIGS. 2A and 2B schematically illustrate longitudinal cross-sections of a handheld body of the system of FIG. 1 according to an example;

FIG. 3A schematically illustrates a perspective view of a shell of the handheld body with a core according to an example;

FIG. 3B schematically illustrates a perspective and partial view of a proximal portion of a shell of the handheld body with a core according to an example;

FIG. 4A schematically illustrates a shell of the handheld body complementary to the shell of FIG. 3A according to an example;

FIG. 4B schematically illustrates a partial view and cross section of the shells of FIGS. 3A and 4A in a coupled state according to an example;

FIG. 5 schematically illustrates a syringe driving system according to an example;

FIGS. 6A and 6B schematically illustrate partial views of the handheld body according to an example with a syringe;

FIG. 7 schematically illustrates a core of the handheld body according to an example and a syringe;

FIG. 8 schematically illustrates a remote actuator according to an example;

FIG. 9 schematically illustrates a force sensor according to an example;

FIG. 10 schematically illustrates a core of the handheld body according to an example and a syringe;

FIG. 11 is a flow chart method according to an example;

FIG. 12 is a flow chart method according to an example;

FIG. 13 is a flow chart method according to an example;

FIG. 14 is a flow chart method according to an example;

FIG. 15 is a flow chart method according to an example.

### DETAILED DESCRIPTION OF EXAMPLES

In the following description, specific details are set forth in order to provide a thorough understanding of the present disclosure.

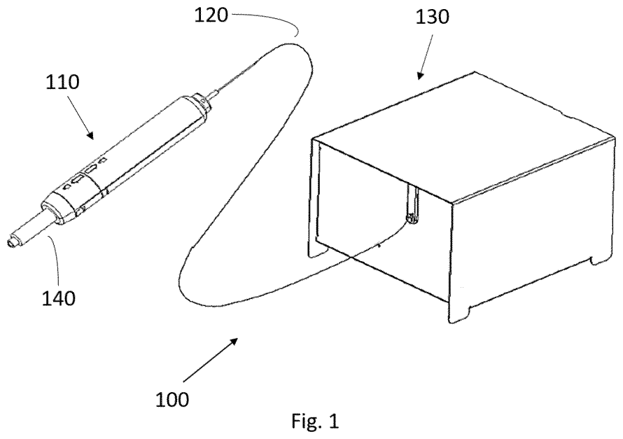

FIG. 1 schematically illustrates a syringe driving system 100 according to an example. The syringe driving system 100 may be a hypodermic syringe driving system 100.

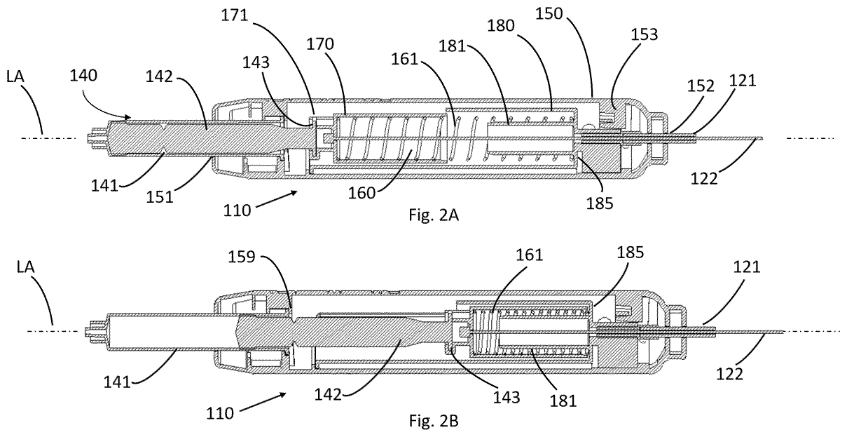

The system 100 comprises a remote actuator 130 operatively connected to a handheld body 110 through a Bowden cable 120. FIGS. 2A and 2B schematically illustrate longitudinal cross-sections of a handheld body of the system of FIG. 1 according to an example. Particularly, the FIG. 2A illustrates the handheld body 110 in an extended position and the FIG. 2B illustrates the handheld body 110 in a retracted position.

The handheld body 110 comprises: a core 160 and a cover 150 to shield the core 160 at least partially, the cover 150 comprises a distal opening 151 to receive a barrel 141 of a syringe 140 and a proximal opening 152 to receive the Bowden cable 120. The core 160 has a movable portion 170 to secure a plunger 142 of the syringe and a fixed portion 180.

As can be seen in FIGS. 2A and 2B, the Bowden cable 120 has a sheath 121 and a wire 122, the wire 122 is displaceable with respect to the sheath 121. The movable and the fixed portion 170, 180 of the core 160 are associated respectively with one of the wire 122 and the sheath 121 in such a way that the movable portion 170 and the fixed portion 180 are displaceable relative to each other along a longitudinal axis LA of the handheld body 110.

In some examples, the wire 122 may be arranged inside the core 160 following a direction of the longitudinal axis LA to contact the movable portion 170. The core and cover may surround the cable inside the handheld body 110.

In some examples, the movable portion 170 and the fixed portion 180 of the core may be arranged in a telescopic fashion along the longitudinal axis LA of the handheld body 110. The latter may allow the movable portion of the core shifting along the longitudinal axis of the handheld body 110 as can be seen in FIGS. 2A, 2B. The longitudinal axis of the fixed portion of the core may coincide with the longitudinal axis LA of the handheld body. The movable portion of the core may be configured to extend e.g., in an extended position, or retract e.g., in a retracted position, from the fixed portion of the core through the sliding over thereof.

In some examples of the systems, the movable and fixed portions of the core may be arranged in a non-telescopic fashion arrangement.

The movable portion 170 and the fixed portion 180 of the core may be slidably joined each other. A relative movement between the movable portion and the fixed portion may be defined in such a way that one of the portions may be introduced into and/or exit from an interior of the other portion, as can be seen in FIGS. 2A and 2B. For example, the movable portion may be introduced into the interior of the fixed portion in a retracted position of the core and the movable portion may exit from the interior of the fixed portion in an expanded position of the core.

The core 160 may have a fixed portion 180 fixedly arranged with respect to the cover 150 and a movable portion 170 movable with respect to the cover 150. The motion may be related to the direction of the longitudinal axis LA.

The sheath 121 may be connected to the fixed portion 180 of the core. This way, a relative movement of the sheath 121 of the Bowden cable with respect to the portion of the core fixed to the cover may be avoided.

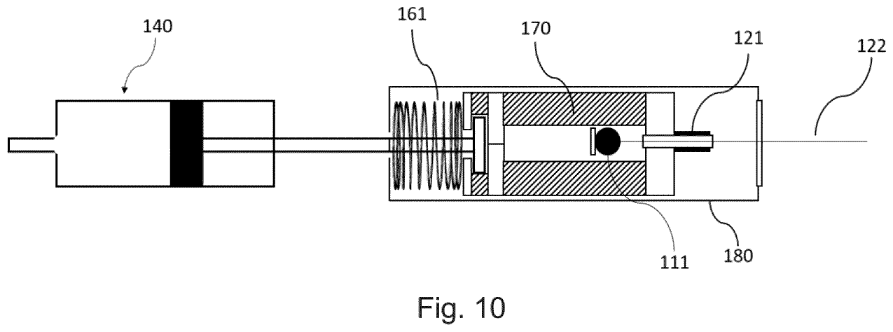

In some examples, the wire 122 may be associated with the fixed portion 180 of the core and the sheath 121 may be associated with movable portion 170 of the core. FIG. 10 illustrates an example of this configuration.

In some examples of the present disclosure, the coupling between the wire 122 and fixed portion 180 of the core 160 may allow the displacement of the movable portion 170 of the core 160 associated to the sheath 121 along the longitudinal axis of the handheld body 110 and relative with respect to the fixed portion 180 of the core 160.

In some examples, the core 160 may be cylindrically shaped. A cylindrically shaped core may facilitate the coupling of a syringe with the actuator system. The cover 150 may be cylindrically shaped as well. Thus, the user, e.g., the medical practitioner, may grab the handheld body 110 in a convenient way.

In some examples, a cylindrically shaped cover may be understood to allocate a cylindrically shaped core configured to couple a syringe. A cylindrically shaped cover may result ergonomically convenient to be handled by a medical practitioner and may result in a less cumbersome syringe actuating process wherein the medical practitioner may be involved in handling a plurality of devices simultaneously.

In some examples the wire 122 may be associated with the fixed portion 180 of the core and the sheath 121 may be associated with movable portion 170 of the core.

As can be seen in FIGS. 2A-2B, the cover 150 and the core 160 may be concentrically arranged about the longitudinal axis LA of the handheld body 110.

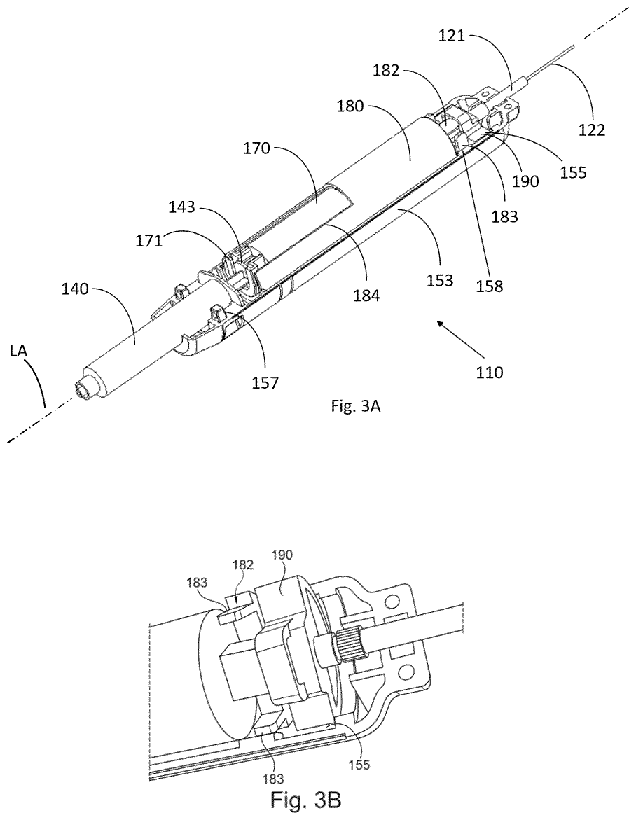

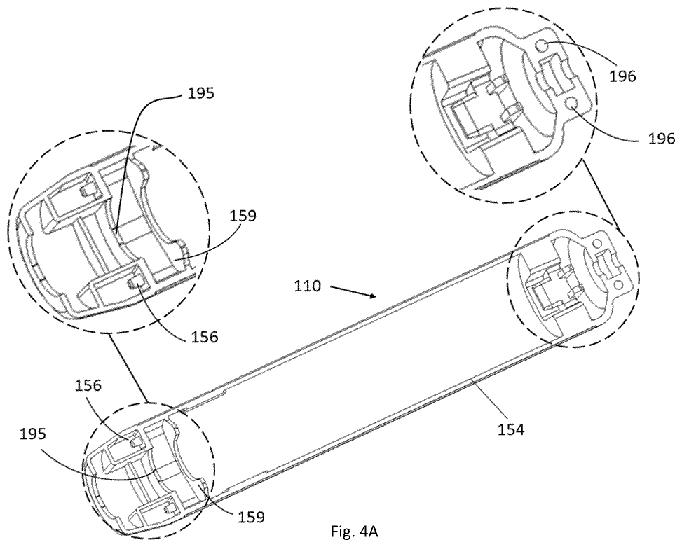

FIGS. 3A and 4 schematically illustrate perspective views of a shell of the handheld body according to respective examples.

In some examples, the cover 150 may be divided in at least two shells 153, 154 along the longitudinal axis LA of the handheld body. The latter can be seen for instance in FIG. 4A. The outline of the shells may be structurally complementary to form a proximal opening and a distal opening when associated.

In some examples of the systems of the present disclosure, the proximal opening 152 of the cover may be understood as the opening resulting from the combination of the outline of the shells 153, 154, the proximal opening 152 may be configured to receive the Bowden cable, and the distal opening 151 may be understood as the opening resulting from the combination of the outline of the shells 153, 154 at the distal end of the handheld body, the distal end may be the most distant portion from the sheath of the Bowden cable.

The two shells 153, 154 may be detachably joined to each other along the longitudinal axis LA of the handheld body 110. In examples, the cover 150 may comprise a securing mechanism to secure the two shells to each other. The securing mechanism may comprise cooperating shapes 156, 157 in such a way that the shapes may cooperate to secure the shells when one shell moves relative to the other shell along the longitudinal axis of the handheld body to a closed status of the handheld body.

The cooperating shapes 156, 157 may comprise a pin arranged in one shell 154 and a hole arranged in the other shell 153. A relative movement of both shells 153, 154 following mutually opposite directions along the longitudinal axis LA to couple the pin and the hole may bring the handheld body 110 to the closed status. A relative movement of both shells 153, 154 following mutually opposite directions along the longitudinal axis LA to disengage the pin and the hole may bring the handheld body 110 to a release status. The closed status may be related to a formed cover 150 for instance a working position where the cover may surround the core and the release status may be related to a separate or open status where the shells 153, 154 are separate and the user can manipulate the core or the syringe.

In examples, the pin may be formed as a L-shaped protrusion and the cooperating shape hole may be configured to allocate the L-shaped protrusion.

The handheld 110 may comprise a tie to join the shells of the cover together. The tie (not shown) may pass through tie holes 196. Tie holes 196 may be arranged at both shells, for instance close an end of the cover close to the proximal opening 152. The tie may pass through tie holes of each shell to join the shells together.

The examples of the systems comprising a securing mechanism comprising a pin and a corresponding hole may reduce or avoid the gap between the shells along the longitudinal axis LA and therefore may reduce the uncertainty in actuating the driving system and so the syringe. This may mean an enhanced accuracy.

In some non-illustrated examples, the cover 150 may have two shells separable or disengageable from each other. The two shells may be separated from each other to access the core without having to slide the two shells relative to each other in the direction of the longitudinal axis. The shells may have a hinge-like mechanism arranged in the longitudinal direction so that the cover may be opened like a book. In other examples, the cover is not hinged and the two shells may be separated.

The cover 150 may comprise a retainer slot 155 to receive a retainer 190. The retainer 190 may be attached to the sheath 121, and the retainer 190 may comprise a bore 191 to pass the wire 122 through. FIG. 3B schematically illustrates a partial view of the handheld according to an example.

As can be seen in FIGS. 3A and 3B, the retainer 190 may comprise a generally flat anchor having a rounded face to be received by the retainer slot 155. The rounded face of the retainer 190 may have a radius or outline configured to match at least partially a semicircle shaped wall of the retainer slot 155.

It is envisaged that the cover may comprise a retainer slot 155 of different shapes and the retainer may comprise a face configured to join the specific shape of the retainer slot. Some examples of the systems of the present disclosure may comprise a retainer slot of rectangular shape, of triangular shape or square shaped. It may be understood that the retainer may comprise a face structurally cooperating with or matching the geometry of the retainer slot of the cover. The geometry may be related to an outline or contour at least partially.

The retainer 190 may be arranged perpendicular to the longitudinal axis LA of the handheld body 110. This may mean that the general flat configuration of the flat anchor may be arranged substantially perpendicular to the longitudinal axis LA. The retainer 190 may be configured to join the slot 155 perpendicularly to the longitudinal axis of the handheld body.

The coupling between the retainer slot and the structurally complementary retainer may reduce unwanted displacement of the fixed portion of the core perpendicularly with respect to the plane of contact between the at least two shells of the cover avoiding the relative dislodgment of the core and the cover of the handheld body.

In some examples according to FIG. 3B, the retainer 190 may comprise a protrusion to constrain a relative rotation of the round shaped retainer 190 with respect to the rounded shaped retainer slot 155. In some examples the retainer may comprise a protrusion to start the relative rotation between the round shaped retainer slot and the complementary round shaped retainer, wherein the relative rotation may start by pushing the protrusion of the retainer. The relative rotation between the retainer slot and the structurally complementary retainer may be understood as perpendicular with respect to the longitudinal axis of the handheld body. FIG. 3B illustrates the retainer 190 displaced relative to the core and about the longitudinal axis. The presence of a retainer slot and a structurally complementary retainer may reduce unwanted movement of the fixed portion of core along the longitudinal axis of the handheld body.

In some examples of the systems of the present disclosure, the fixed portion 180 of the core 160 may comprise a fastener 190 comprising a bore such that the wire may pass through the bore and the sheath may be fastened to the core.

The core 160 may comprise a main spring 161 arranged between the movable portion 170 and the fixed portion 180 such that the movable portion 170 may be biased away from the fixed portion 180. The latter may mean that a relative approach between the movable portion and the fixed portion can cause a contraction of the main spring, i.e. the retracted position. The main spring 161 may be compressed and so it may bias the movable portion to the expanded position.

The characteristics of the main spring 161 may be chosen according to the dimensions of the handled body and/or the syringe. In this way the spring may vary the force exerted on the syringe plunger as well as the stroke.

The systems of the present disclosure may comprise a main spring or an elastic body configured to recover its original shape when released after being distorted, arranged between the movable portion and the fixed portion of the core, and configured to exert a force in a direction contrasting the direction of the force applied by the remote actuator through the Bowden cable.

In some examples, the movable portion may approach the fixed portion driven by a relative displacement of the wire and the sheath and contracting the main spring. A relative displacement of the wire and the sheath in an opposite direction may allow the main spring to expand and bias the movable portion away from the fixed portion. In examples, the main spring may be compressed when the movable portion moves away from the fixed portion and may expand when the movable portion approaches the fixed portion.

The fixed portion 180 of the core may have a sleeve 181 about the wire 122 at least and the main spring 161 may be a coil spring arranged about the sleeve 181. The sleeve 181 may be configured to partially surround or wrap the wire 122 and the main spring may be configured to encircle the sleeve at least partially. The sleeve may set the wire apart from the main spring. The sleeve may avoid the interference of the main spring to actuating of the wire. Examples of unnecessary interactions between the spring and the wire include but not are limited to the contact due an excessive force exerted by the spring causing a displacement of the spring in a perpendicular axis with respect to the longitudinal axis of the handheld body where the wire and the spring are distributed. The examples of the attached figures disclose systems where the main spring is disposed around the longitudinal axis LA.

In some examples, the fixed portion 180 of the core may be detachably coupled to the cover 150. This way, the cover 150 may be configured as a single-use or disposable part. The core 160 may be reusable or disposable. The cover 150 which may be an outermost part of the handheld body 110 may be exposed to any kind of harmful substances like germs, pollutants or contamination, so a single use configuration may provide an enhanced hygiene. A reusable core 160 may reduce material waste after operation. This may mean reducing impact on the environment and even reducing operation costs.

The fixed portion may comprise a releasable clip 182 in such a way that the fixed portion is releasable secured to an inner wall of the cover 150. The releasable clip 182 may be integrally formed with the rest of the fixed portion 180. In the illustrate examples the releasable clip is arranged in an end of the fixed portion 180 in such a way that the releasable clip is disposed close to the proximal opening 152. Some examples of the systems of the present disclosure may comprise a releasable clip arranged transverse to the inner wall of the cover, with respect to the longitudinal axis LA. By releasing the clip 182, core may be detached from the cover.

FIG. 3B schematically illustrates a releasable clip 182. The releasable clip may have a tab 183 to engage a ledge 158. The ledge may be arranged in the inner wall of the cover 150. The number of tabs and ledges may vary.

The fixed portion 180 may be detachably coupled to the cover in such a way that the fixed portion 180 of the core may be axially blocked with respect to the cover 150. This may mean that there is substantially no relative displacement along the longitudinal axis LA between the fixed portion 180 and the cover 150.

In the examples of the systems comprising the releasable clip 182, the tab 183 thereof to engage the ledge 158 of the cover may block the fixed portion 180 of the core axially with respect to the cover 150 about the longitudinal axis and forcing the fixed portion of the core like being integral to a shell of the cover.

In some examples of the systems of the present disclosure, the releasable clip may be meant to fit into a shell of the cover configured to be detachably coupled to the core, the releasable clip may be configured to fix the core to the cover, the releasable clip being arranged between the fixed portion of the core and the retainer. In some examples of the present disclosure, the releasable clip may limit the axial movement between the fixed portion of the core and the shell of the cover to which the core is fixed. The releasable clip 182 may limit a rotational movement of the fixed portion relative the cover and about the longitudinal axis.

The distal opening 151 may comprise a seal to surround the barrel 141 of the syringe. The seal may avoid or at least reduce a flow of a fluid in the area defined between the distal opening 151 and the barrel 141. The seal may be arranged over the distal opening, for instance a seal formed by parts disposed in each shell 153, 154. Examples of the seal of the present disclosure may comprise a bendable seal.

The size of the distal opening may be chosen according to the size of the barrel to be received. Thanks to the bendable seal, the same distal opening may accommodate barrels of different diameters. The flexible and bendable characteristic of the seal makes it possible to adapt to different diameters.

The cover 150 may comprise a ridge 195 disposed between the distal opening 151 and the core 160 to support the barrel 141, the ridge 195 may comprise a rounded region to match at least partially a side contour of the barrel.

In examples, each of the shells of the cover may comprise respectively a holding pad 159 protruding from an inner face of the shell. The holding pads 159 of each shell may be arranged at different points along the longitudinal axis of the handheld body such that a barrel flange of the syringe may be sandwiched between the holding pads. In a closed status, the holding pads 159 of each shell may be disposed at different points along the longitudinal axis, so the flange of the barrel may be sandwiched between a pair of pads 159, each pad from different shell. The sandwich configuration may be caused by the relative movement of the shells against each other to close or form the cover.

In some examples, the holding pads 159 of each shell may be configured to fix the barrel flange of the syringe in between of the holding pads. An example of the systems of the present disclosure may comprise a plurality of holding pads protruding from an inner face of a shell of the cover, so that the distance between the holding pads of the shell may be arranged to host the barrel flange of the syringe at a predefined position.

In examples, one of the shells may comprise a holding pad 159 while the other shell may comprise an abutment surface 59 to sandwich the flange 143 of the syringe in a closed status, e.g. a coupled state. This feature can be seen in FIG. 4B where the shells 110 of FIGS. 3A and 4A are illustrated in a coupled state according to an example. The examples of the system having a pad 159 and an abutment surface 59 may act in a similar way as the examples having a pair of pads 159 mutatis mutandis. Although the FIG. 4B illustrates the case of a pad and an abutment surface, a similar configuration may be achieved with a pad 159 at each shell 110. In the latter case, a pad 159 would be located instead of the abutment surface 59.

Manufacturing tolerances may lead to gaps or clearances between a syringe and the system 100, and particularly between the syringe and the handheld body. Gaps or clearances may cause that forces are not transmitted in a linear and homogeneous way between the system 100 and the syringe, but that the force may be transmitted abruptly. The latter may cause a backlash which has to be avoided or at least reduced in order to guarantee a smooth operation of the system.

Although the manufacture or assembly of the components of the system of the present disclosure could generate gaps that may cause backlash, at the one hand such gaps may allow the coupling between the shells, and at the other hand, the gaps may avoid the shells being forced against each other avoiding undesired jamming.

It is understood that, for instance, when a remote actuator is actuated to move the plunger of a syringe associated to the movable portion of the core from 0 mm to 10 mm, a tolerance equal to ±0.2 mm between elements of the handheld body 110 or between the handheld body 110 and the syringe may force the plunger to move with respect to the barrel between 0 mm and 9.8 mm, i.e. different numbers. A gap that could be present between the system and the syringe would limit the distance travelled inside the plunger to a length very similar to the uncertainty of such a gap. The presence of the holding pad 159 may secure the syringe, particularly the barrel, so that a relative movement between the movable portion and the fixed portion may cause a substantially similar displacement between the plunger and the barrel.

In the present disclosure, the tolerance may be understood as the amount of variation that is allowed in a measurement in the manufacture or assembly of a component. The tolerance may depend on the material and on the manufacturing process. In some examples of the systems of the present disclosure, the shells may be manufactured in plastic material. It is commonly known that a large-scale plastic part production may lead uncertainty and producers may define a certain tolerance. In the field of large-scale plastic production, a tolerance of ±0.1 mm could be understood as accepted, and the thickness of the barrel flange may have a tolerance of ±0.1 mm. The combination of the tolerance of the thickness of the syringe flange and of the shells may be meant equal to ±0.2 mm of gap and may cause backlash.

When the securing mechanism is in a closed status of the handheld body, the cooperating shapes of the securing mechanism may facilitate pressing the shells against each other along the longitudinal axis. Thus, the pads 159 or a pad and a complementary surface of the other shell may be pressed against each other with the barrel flange in-between. As the pads 159 or the pad and the complementary surface of different shells may apply force to the barrel flanges, the clearance between the barrel flanges and the pad may be avoided or at least reduce. Therefore, the backlash may be avoided or reduced due to any gap that may exist between the barrel flange and the cover.

When the handheld body is in a closed status, the cooperating shapes of the securing mechanism may facilitate pressing the shells against each other along the longitudinal axis and thus eliminating the backlash due to any gap that may exist between the syringe wing and the cover.

Furthermore, the uncertainty that may exist about the positioning of the barrel flange when the plunger moves in the direction of the longitudinal axis from a position close to the proximal opening to a new position far from the proximal opening, may be avoided or at least reduced. It may be understood that when the plunger of a syringe associated to the movable portion of the core is at the minimum distance from the distal opening, such distance may be greater than the possible uncertainty that may exist.

The fixed portion 180 of the core may comprise a stopper 185 to define an end of stroke of the movable portion 170 towards the proximal end 152 of the cover. This way, the displacement of the movable portion of the core may be controlled and may contribute as protection system in the case wherein the remote actuator could apply an excessive force to the wire 122. The minimum distance between the movable portion of the core and the distal opening of the cover may be guaranteed by the presence of a stopper configured to block the movable portion of the core when the movable core is at the closest position from the distal opening. In some examples of the systems of the present disclosure, a stopper mounted in the internal wall of the fixed portion of the core and configured to block the movable portion of the core may arrest the movement towards the proximal opening when the movable portion of the core reaches the minimum distance therefrom.

The movable portion 170 of the core may comprise a flange notch 171 to receive a plunger flange 143 of the syringe. The flange notch 171 may be arranged at one end of the movable portion towards the distal end 151, taking the longitudinal axis LA direction. The flange notch 171 may be substantially U-shaped.

In some examples, the flange notch 171 and the plunger flange 143 may have complementary shapes, so as to ensure coupling between the two parts, reducing or eliminating clearances and play therebetween. In some cases, the tolerance between the two parts may be almost negligible.

The fixed portion 180 of the core may have a side elongated cut-out 184 arranged to insert the plunger 142 of the syringe in an expanded status. The expanded status may be seen in FIG. 2B. The expanded status of the syringe may indicate that the plunger thereof is substantially at the maximum extension. The cut-out may be disposed in a distal end of the fixed portion.

The cut-out 184 may be configured as an indentation. The length of the cut-out (along the longitudinal axis) may allow to insert and to extract the syringe from the handheld body 110 for instance when the syringe is in the expanded status. The width of the cut-out (substantially perpendicular to the longitudinal axis) may allow to pass through the width of a barrel. The user can do it when the shells are not secured each other.

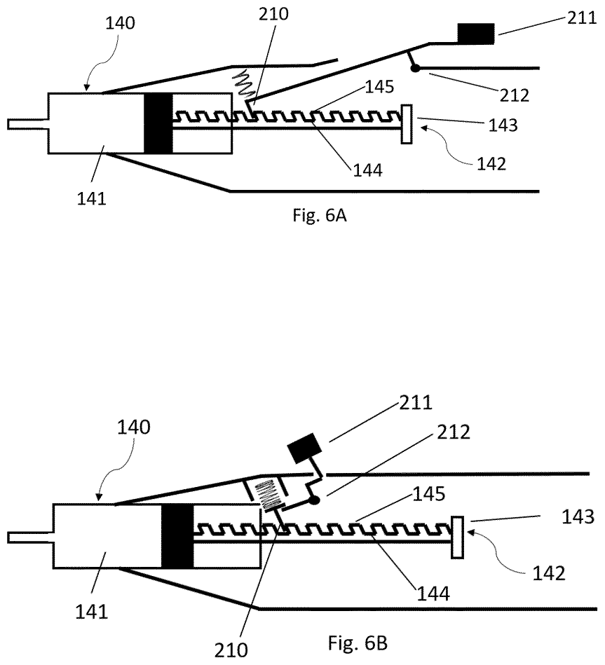

According to some examples, as those illustrated in FIG. 7, the system may comprise a spring-loaded wedge 163 rotatably attached to the movable portion 170 of the core 160, the wedge being able to move from a retracted status to allow a relative displacement between the movable portion 170 and the fixed portion 180 of the core 160, to an expanded status biased by a wedge spring 162 to block the relative displacement between the movable portion and the fixed portion. FIG. 7 shows an example of the core of the system 100 in which the movable portion 170 and the main spring 161 are in contact with a spring-loaded wedge 163 in a retracted status to allow the relative displacement of the core. In case of fault of the system 100 or damage of the wire 122, the spring-loaded wedge 163 may move to an expanded status to block the relative displacement of the core. Wedges 163 may be kept in a retracted status by a force exerted by the main spring against the movable portion. If the wire is broken or the like, the movable portion may move away from the main spring and the wedges may be released. Thus, the wedges may be biased by the wedge spring 162 to the expanded status.

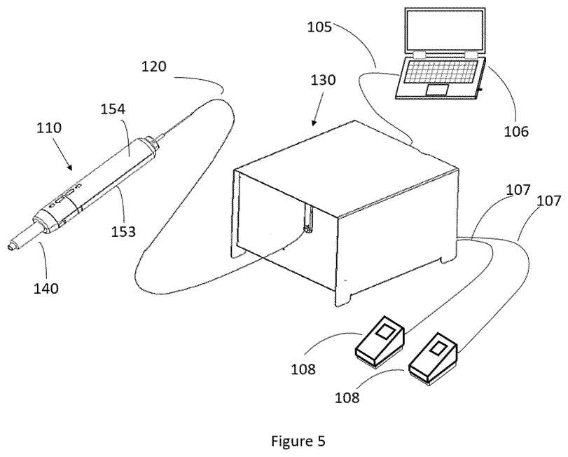

FIGS. 6A and 6B schematically illustrate partial views of a syringe 140 and the handheld 110 according to an example comprising a spring-loaded pawl. Some examples of the system 100 related to FIGS. 6A and 6B may comprise a spring-loaded pawl 210 to engage a number of teeth 145 arranged along the plunger 142 of the syringe 140 in such a way that the plunger 142 may be blocked, and a button 211 operatively connected to the pawl 210 through a hinge point 212 in such a way that the pawl 210 may be disengaged from the teeth 145 upon actuation of the button 211.

According to some examples, the button 211 may be placed on the cover 150. The cover 150 may comprise a button 211 arranged in a portion of the external wall and operatively connected to a spring-loaded pawl 210 arranged in a portion of the internal wall of the cover 150. If the button 211 is not pressed, the plunger is not allowed to move. As a result of such configuration the risk of undesired actuation of the system 100 may be reduced.

According to yet another example, the spring-loaded pawl 210 may be configured to engage a plunger comprising a plurality of equidistant teeth.

FIGS. 6A and 6B show a syringe 140 for the syringe driving system 100. The syringe 140 that may comprise a number of teeth 145 and plunger notches 144 along a plunger 142, the teeth 145 being configured to receive the pawl 210 such that a ratchet mechanism is defined. In some examples of the syringe 140 comprising a number of teeth 145 configured to lock or activate the actuating of the movable portion 170 of the core 160 detachably coupled to the syringe 140 through the pawl 210.

In FIG. 10 it can be seen that the main spring 161 is arranged between the movable part 170 and the fixed part 180. In this example, the main spring is located on the side closest to the distal opening. When the main spring is compressed, the syringe plunger is in its retracted position and when the main spring is in its most extended position, the plunger is also in its extended position. The wire 122 of the Bowden cable 120 is attached to the cover 150 through a pin 111. In this way the relative sliding between the wire 122 and the sheath 121 may cause a relative movement between the movable part 170 and the fixed part 180 which in turn may cause actuation of a syringe attached to the movable part 170. In case of failure, for example, breakage of the Bowden cable, the spring 161 will tend to its extended position and thus also the plunger.

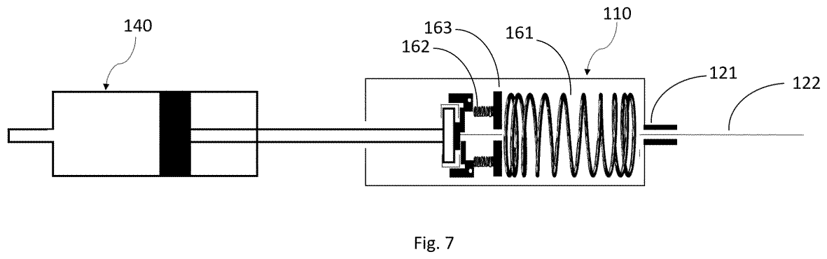

In some examples, the remote actuator 130 may comprise a 1 131 to drive a pulley 132 to wind and unwind the wire, and the motor 131 and the pulley 132 may be connected through a gearbox 133. By winding or unwinding the wire, a relative displacement between the wire and the sheath may be caused. In FIG. 8, some parts of the remote actuator 130 can be seen.

The sheath 121 may be fastened to the actuator 130 through a fastener 134. The wire 122 may be fastened to the pulley 132.

Some examples of the remote actuator may comprise a motor 131 configured to move forward and backward the movable portion 170 of the core 160 of the handheld body 110 through the wire 122, such that the movable portion 170 of the core 160 may shift a plunger of a syringe 140 configured to be received along the longitudinal axis of the handheld body 110, wherein unwinding the wire 122 shifts the plunger of the syringe 140 connected to the movable portion 170 of the core 160 forward from the proximal portion close to the proximal opening 152 to a distal portion close to the distal opening 151 and winding the wire 122 shifts the plunger connected to the movable portion 170 of the core 160 back to a position close to the proximal opening 152. In some examples, the operation may be the opposite. Some examples of the systems of the present disclosure may comprise brushless motors.

In some examples, the remote actuator 130 may comprise a control unit to control the operation of the motor 131.

According to some examples, the system may comprise a remote actuator 130 with a control unit. The control unit may comprise a receiving module to receive operational data; a determining module to determine working parameters of the motor 131 based on the received operational data, wherein the working parameters may comprise a duration, a speed and a direction of rotation; an activating module to activate the motor 131 based on the determined working parameters.

The control unit may manage a series of steps to be sequentially executed by the motor. The steps may be organized in a sequence. The sequence may be predefined or may be generated based on an input from the user. Some examples of sequences may comprise starting the execution of a task, keeping active the execution of a specific task with high accuracy, and stopping the execution of the sequence. Non exhaustive examples of specific tasks may be intended to be e.g., moving forward the movable portion of the core with controlled speed, moving back the movable portion of the core with a controlled force.

The examples involving a sequence of steps may reduce the effort for the medical practitioner in holding the device or in applying a force to the core of the handheld body.

In some examples of the systems of the present disclosure, the control unit may further control the speed of the sliding movable portion of the core. The control on the speed of the sliding movable portion of the core may preserve the plunger of the syringe from being moved extremely quickly. Furthermore, this control on the speed may guarantee the preservation of the surrounding tissue of the extracted fluid sample.

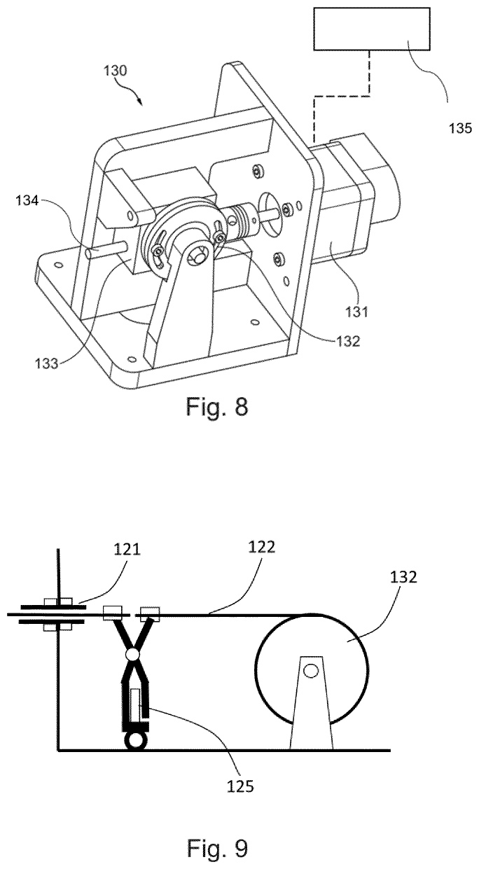

Some examples of the systems may comprise a force sensor 125 to sense a force applied to the wire 122, see for instance FIG. 9. The force sensor 125 may be associated to the remote actuator 130. As a result of such configuration, sensing the force applied to the wire 122 may guarantee the system 100 being actuated in accord with the instructions of the control unit. According to some examples, the force sensor 125 may be associated to the remote actuator 130 in the proximity of the pulley 132.

According to some examples, the system 100 may comprise a confirmation switch in electrical or data communication with the control unit to operate the motor 131.

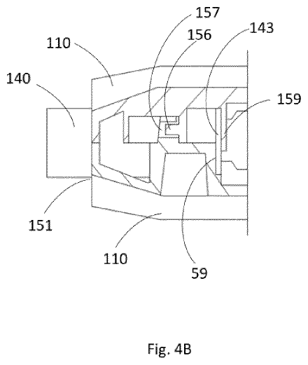

The confirmation switch may be a pedal 108, as seen in FIG. 5. The examples comprising a pedal 108 may facilitate operating the control unit wherein the instructions of the control unit may be required to be confirmed trough the confirmation pedal 108. Some examples comprising two pedals 108 may result in a further protection in which a first pedal may confirm the instructions to actuate the system in a direction and the second pedal may confirm the instructions to actuate the system in an opposite direction with respect to the direction of the first pedal.

Following with FIG. 5, the system 100 according to some examples, may comprise an input device 106, e.g. a computer or the like that may be in data communication with the remote actuator 130 through a cable 105 or the like. The user may introduce operational data through the input device. The pedals 108 are in data or electrical communication with the remote actuator 130 through cables 107. In some examples, the different parts of the system 100 may be in data communication wirelessly.

The input device 106 may be integrally formed with the remote actuator.

The system or a part of the system may be configured to be mobile and carried on a wheeled stand, for example a wheeled pedestal (not illustrated). In one example the remote actuator and the input device may be located on the mobile stand. The handheld body may also be housed in some retaining device suitable for that purpose in the mobile stand.

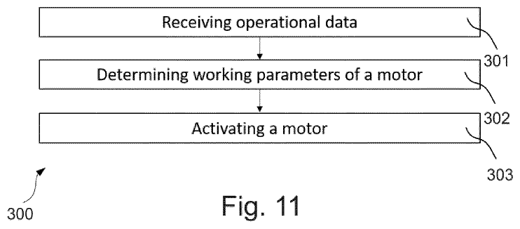

The present disclosure also relates to a method 300 for driving a hypodermic syringe driving system 100 according to any of herein disclosed examples. The method 300 comprises: receiving operational data 301; determining working parameters of the motor based on the received operational data 302; wherein the working parameters comprise at least one of duration, speed and direction of rotation; activating the motor based on the determined working parameters 303. FIG. 11 shows an example of method 300 comprising receiving operational data, determining a working parameter of a motor and activating the motor.

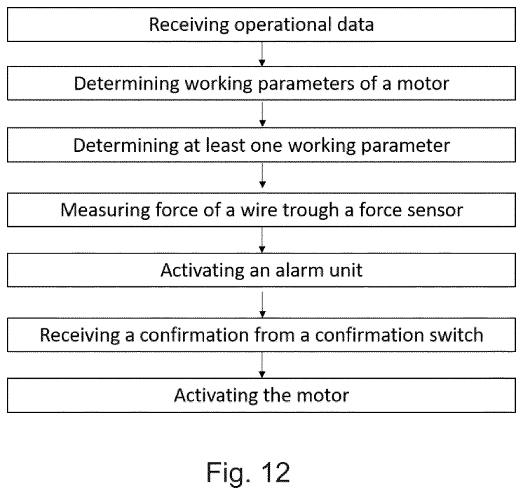

FIG. 12 shows a non-limitative example of a method according to the present disclosure.

The order of the steps of the herein disclosed methods may differ, e.g., some steps may be excluded, added, or changed in position.

In some examples, the duration may be related to the duration of the relative motion between the movable portion and the fixed portion of the core. The speed may be related to the speed at which the relative motion between the movable portion and the fixed portion of the core may occur. The direction may be related to the direction of the relative motion between the movable portion and the fixed portion of the core, relative to the longitudinal axis LA.

In examples, relative motion or movement between the movable portion and the fixed portion may be performed continuously or step-wise. A continuous or step-wise motion may be determined by the control unit depending on the operational data.

The operational data may comprise data entered through a user interface. In examples, the input device 106 may comprise the user interface. In some examples, the user interface may be a touch screen, buttons or the like.

In some examples, the operational data may comprise at least one of a volume to be applied, flow rate, and direction of displacement of the movable portion relative to the fixed portion of the core. The operational data may be received by the control unit.

In examples, the volume may be the volume of a hypodermic substance to be administered or the volume of a sample to be taken. The flow rate may be the flow rate at which a hypodermic substance is to be administered or a sample is to be taken. The direction of displacement may be the direction associated with the relative displacement to generate a suction or aspiration force within the barrel of the syringe, e.g., to take a sample, or to generate a pressure force within the barrel of the syringe to expel the hypodermic substance from the barrel.

In some examples, the motor may be associated with the pulley and the pulley may be associated with the Bowden cable. By determining the working parameters, the operation of the motor may be controlled and thus the relative motion between the wire and the sheath of the cable may be controlled. As explained above, the relative movement of the wire and sheath may cause relative movement between the fixed portion and the movable portion of the core. This relative movement may cause displacement of the plunger of a syringe relative to the barrel of the syringe, the syringe being received by the handheld body. Features of the relative movement may be adjusted, for instance as determined working parameters based on the received operational data.

In some examples, the method 300 may comprise receiving confirmation data from the confirmation switch before activating the motor.

Some examples of the methods of the present disclosure may execute the confirmation of a series of instructions comprising displacing the movable portion of the core along the longitudinal axis of the handheld body towards the proximal opening, waiting a first predetermined time, displacing the movable portion of the core towards the distal opening, waiting a confirmation command, displacing the movable portion of the core towards the proximal opening, waiting a second predetermined time or a confirmation command and displacing the movable portion of the core towards the distal opening.

The operational data may comprise data received from the force sensor 125 associated to the wire 122. This way, the tension in the wire may be verified and taken into account. The tension of the wire may be computed by the control unit based on the readings from the force sensor. Tension value may be compared with a tension threshold. The tension threshold may refer to a predefined range of tension values. In examples, if the read tension value is within the range, the motor may be operated. Otherwise, the operation of the motor may be prevented, or the user may be warned.

The actuator 130 may comprise an alarm unit in electrical communication with the control unit. In such a case the method may comprise activating the alarm unit based on data received from the force sensor. The alarm unit may generate an alarm such as audio, visual or haptic alarm. The alarm unit may be triggered by the control unit in case the read tension value is out of the range of tension values above.

By way of example, a breakage of the wire may cause the read tension value to be below a lower limit range value. The alarm unit may be triggered. A jammed or stuck part of the system, such as the movable portion relative to the fixed portion may cause the read tension value to be above a high limit range value. The alarm unit may be triggered accordingly.

For example, if the tissue in which a substance is to be injected is significantly hard, e.g. fibrosis, the substance may be retained in the barrel. The tissue may prevent or limit the exit of the syringe. At the same time, the motor may be running to allow relative movement between the fixed part and the movable part. Depending on the configuration, the spring may be driving the relative displacement between the fixed part and the movable part. Consequently, there may be a decrease in the Bowden cable tension value. The pressure sensor may detect the pressure value, and the control unit may process the information. The alarm unit may be triggered as discussed above. In an example, the user interface may show a red light or the like.

The motor may be activated following a sequence of working parameters or sets of working parameters, i.e., a sequence of the determined working parameters that may be chosen by the actuator 130 e.g., the control unit thereof. The sequence may be chosen among different predefined sequence templates. The sequence templates may be chosen by the control unit thereof by comparing the received working data with predefined working data associated with the templates. In examples, a sequence template may be received as working data.

Examples of sequence templates may comprise biopsy and/or injection.

Before performing an action within the sequence of working parameters, any of the herein disclosed examples of the methods may comprise waiting for a confirmation command.

In examples, a sequence of set of working parameters may comprise displacing the movable portion of the core along the longitudinal axis of the handheld body towards the proximal opening, waiting a first predetermined time, displacing the movable portion of the core towards the distal opening, displacing the movable portion of the core towards the proximal opening, waiting a second predetermined time and displacing the movable portion of the core towards the distal opening.

According to an example, a sequence of working parameters or sets of working parameters may comprise displacing the movable portion of the core along the longitudinal axis of the handheld body towards the distal opening, waiting a third predetermined time, and displacing the movable portion of the core towards the proximal opening.

In some examples, the sequence of working parameters or sets of working parameters may be determined based on operational data or sets of operational data received in a particular sequence.

According to examples, a sequence of working parameters or sets of working parameters may comprise displacing the movable portion of the core along the longitudinal axis of the handheld body towards the proximal opening, waiting a fourth predetermined time, and displacing the movable portion of the core towards the distal opening.

The actions performed through the above sequences may be implemented in a different order.

The present disclosure also relates to a method 400 for carrying out a fine needle aspiration biopsy using a hypodermic syringe driving system according to any of herein disclosed examples and a syringe. The method 400 may comprise:
receiving operational data 401. Features regarding the operational data may be similar to or the same as those related to the herein disclosed methods;
determining working parameters of the motor based on the received operational data 402, wherein the working parameters comprise duration, speed and direction of rotation;
inserting a needle of the syringe into a body of a patient 403,
activating the motor to displace the movable portion with respect to the fixed portion, generating a suction force in the barrel of the syringe 404. The suction force may be generated by displacing the movable portion towards the proximal opening of the handheld body along the longitudinal axis of the handheld;
obtaining a fluid sample of the patient 405.

In some examples, method 400 may comprise activating the motor to displace the movable portion with respect to the fixed portion before inserting the needle into the body of the patient. This way, air present in the barrel may be expelled. Thus, the safety of the operation may be enhanced.

In some examples of the method 400, activating the motor for generating a suction force may require various steps that may comprise displacing the movable portion of the core along the longitudinal axis of the handheld body towards the proximal opening to reach the maximum extended status of the plunger of the syringe, waiting a first predetermined time, displacing the movable portion of the core towards the distal opening to expel the air present in the barrel, waiting a confirmation command, inserting a needle of the syringe into a body of a patient, displacing the movable portion of the core towards the proximal opening to execute a suction of a hypodermic fluid, waiting a second predetermined time or a confirmation command, remove the needle of the syringe from the body of a patient and displacing the movable portion of the core towards the distal opening to expel the fluid from the syringe.

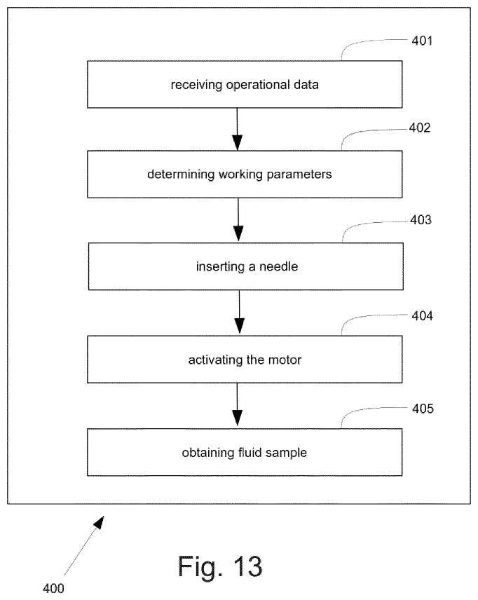

An example of method 400 can be seen in FIG. 13.

The present disclosure also relates to a method 500 for injecting a substance into a patient using a hypodermic syringe driving system according to any of herein disclosed examples and a syringe. The method 500 may comprise:
receiving operational data 501. Features regarding the operational data may be similar to or the same as those related to the herein disclosed methods;
determining working parameters of the motor based on the received operational data 502, wherein the working parameters comprise duration, speed and direction of rotation;
activating the motor to displace the movable portion with respect to the fixed portion 503;
inserting a needle of the syringe into the body of the patient 504;
activating the motor to displace the movable portion with respect to the fixed portion to push the plunger of the syringe 505;
expelling a substance through the needle 506.

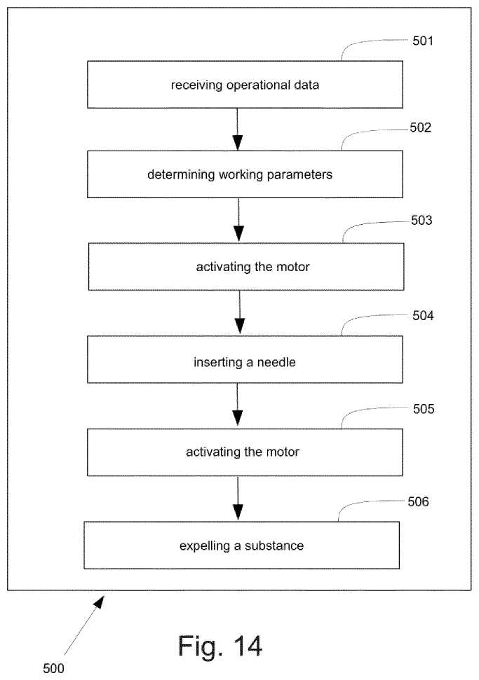

An example of method 500 can be seen in FIG. 14.

The present disclosure also relates to a method 600 for injecting a substance and carrying out a fine needle aspiration biopsy using a hypodermic syringe driving system according to any of herein disclosed examples and a syringe. The method 600 may comprise:
receiving operational data 601. Features regarding the operational data may be similar to or the same as those related to the herein disclosed methods;
determining working parameters of the motor based on the received operational data 602, wherein the working parameters comprise duration, speed and direction of rotation;
activating the motor to displace the movable portion with respect to the fixed portion 603;
inserting a needle of the syringe into a body of a patient 604;
activating the motor to displace the movable portion with respect to the fixed portion to push the plunger of the syringe to inject an oncolytic virus substance into the body of the patient 605;
activating the motor to displace the movable portion with respect to the fixed portion to pull the plunger of the syringe to obtain a fluid sample of the patient 606.

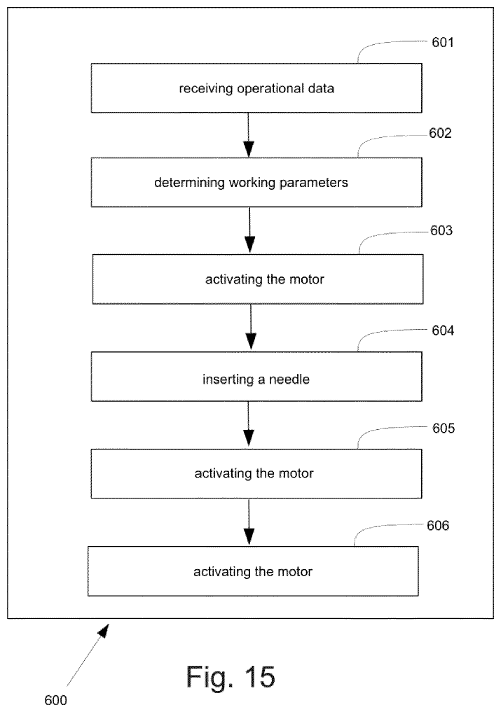

An example of method 600 can be seen in FIG. 15.

The method 600 may be implemented in cases involving e.g. the treatment of the thyroid or lymph-nodes. However, other cases may be envisaged.

According to one example, a user may enter operational data through the user interface. The operational data may include at least one of volume, such as the volume of the substance to be injected or the volume of the sample to be extracted, the flow rate, i.e., volume injected or extracted in a given time, and the direction of displacement of the plunger in the direction of the longitudinal axis, i.e. the direction in which the movable portion moves relative to the fixed portion along the longitudinal axis. The user may enter a value for each of the above data or may enter a set of values, where in each set there is at least one of volume, flow rate and/or direction.

The control unit that has received the operational data may determine the working parameters of the motor. The working parameters of the motor are determined to achieve at least one of a total or partial angular displacement of the pulley, the angular speed of displacement of the pulley and/or the angular direction of the angular displacement of the pulley, for example clockwise or counterclockwise. In the case where the operational data have been entered as sets and/or following a particular sequence, the working parameters are determined for each set and/or following a particular sequence.

The control unit may wait to receive a confirmation command before actuating the motor. The confirmation command may be associated with a particular switch for a direction of displacement of the movable portion. If the confirmation command is received, the control unit may send the corresponding command to actuate the motor. If there is a sequence of working parameters, the control unit may wait to receive the confirmation command for each set. In this way the user decides when the working parameters that govern the operation of the motor are implemented. The user may perform tasks and when he/she has finished and wants the system to start up, actuate the confirmation switch.

The system may inform the user of the actions that are implemented through the user interface. The user can know the operation to be executed and for which the system requires confirmation.

For reasons of completeness, various aspects of the present disclosure are set out in the following numbered clauses:
Clause 1. A hypodermic syringe driving system comprising a remote actuator operatively connected to a handheld body through a Bowden cable, wherein the handheld body comprises:
   a core and a cover to shield the core at least partially, the cover comprises a distal opening to receive a barrel of a syringe and a proximal opening to receive the Bowden cable;
   wherein the core has a movable portion to secure a plunger of the syringe and a fixed portion,
   wherein the Bowden cable has a sheath and a wire, the wire being displaceable with respect to the sheath, the movable and the fixed portion of the core being associated respectively with one of the wire and the sheath in such a way that the movable portion and the fixed portion are displaceable relative to each other along a longitudinal axis of the handheld body.
Clause 2. The system according to clause 1, wherein the movable portion and the fixed portion of the core are arranged in a telescopic fashion along the longitudinal axis of the handheld body.
Clause 3. The system according to any of clauses 1-2, wherein the sheath is connected to the fixed portion of the core.
Clause 3 may avoid any relative movement of the sheath of the Bowden cable with respect to the portion of the core fixed to the cover of the handheld body.
Clause 4. The system according to any of clauses 1-3, wherein the movable portion and the fixed portion of the core are slidably joined each other.
Clause 5. The system according to any of clauses 1-4, wherein the cover comprises a retainer slot to receive a retainer, the retainer being attached to the sheath, wherein the retainer comprises a bore to pass the wire through.
Clause 6. The system according to clause 5, wherein the retainer comprises a generally flat anchor having a rounded face to be received by the retainer slot.
Clause 7. The system according to clause 6, wherein the anchor is arranged perpendicular to the longitudinal axis of the handheld body.
Clause 8. The system according to any of clauses 1-7, wherein the core comprises a main spring arranged between the movable portion and the fixed portion such that the movable portion is biased away from the fixed portion.
Clause 9. The system according to any of clauses 1-8, wherein the fixed portion of the core has a sleeve about the wire and the main spring is a coil spring arranged about the sleeve.
Clause 10. The system according to any of clauses 1-9, wherein the cover is divided in at least two shells along the longitudinal axis of the handheld body.
Clause 11. The system according to clause 10, wherein the two shells are detachably joined to each other along the longitudinal axis of the handheld body.
Clause 12. The system according to any of clauses 10-11, wherein the cover comprises a securing mechanism to secure the two shells to each other, the securing mechanism comprising cooperating shapes in such a way that the shapes cooperate to secure the shells when one shell moves relative to the other shell along the longitudinal axis of the handheld body to a closed status of the handheld body.
Clause 13. The system according to clause 12, wherein the pin is formed as a L-shaped protrusion.
Clause 14. The system according to any of clauses 1-13, wherein the fixed portion of the core is detachably coupled to the cover.
Clause 15. The system according to any of clauses 1-14, comprising a releasable clip in such a way that the fixed portion is releasably secured to an inner wall of the cover.
Clause 16. The system according to clause 15, wherein the releasable clip has a tab to engage a ledge.
Clause 17. The system according to any of clauses 1-16, wherein the fixed portion of the core is axially blocked with respect to the cover.
Clause 18. The system according to any of clauses 1-17, wherein the distal opening comprises a seal to surround the barrel.
Clause 19. The system according to any of clauses 1-18, wherein the cover comprises a ridge disposed between the distal opening and the core to support the barrel, the ridge comprising a rounded region to match at least partially a side contour of the barrel.
Clause 20. The system according to any of clauses 1-19, wherein each of the shells of the cover comprise respectively a holding pad protruding from an inner face of the shell, the holding pads of each shell being arranged at different points along the longitudinal axis of the handheld body such that a barrel flange of the syringe is sandwiched between the holding pads.
Clause 21. The system according to any of clauses 1-20, comprising a tie to join the shells of the cover together.
Clause 22. The system according to any of clauses 1-21, wherein the movable portion of the core comprises a flange notch to receive a plunger flange of the syringe.
Clause 23. The system according to any of clauses 1-22, wherein the core has the fixed portion fixedly arranged with respect to the cover and the movable portion movable with respect to the cover.
Clause 24. The system according to any of clauses 1-23, wherein the fixed portion of the core has a side elongated cut-out arranged to insert the plunger of the syringe in an expanded status.
Clause 25. The system according to clause 24, wherein the cut-out is disposed in a distal end of the fixed portion.
Clause 26. The system according to any of clauses 1-25, wherein the fixed portion of the core comprises a stopper to define an end of stroke of the movable portion towards the proximal end of the cover.
Clause 27. The system according to any of clauses 1-26, wherein the core is cylindrically shaped.
Clause 28. The system according to any of clauses 1-27, wherein the cover is cylindrically shaped.
Clause 29. The system according to any of clauses 1-28, wherein the cover and the core are concentrically arranged about the longitudinal axis of the handheld body.
Clause 30. The system according to any of clauses 1-29, wherein the remote actuator comprises a motor to drive a pulley to wind and unwind the wire, and the motor and the pulley are connected through a gearbox.
Clause 31. The system according to any of clauses 1-30, wherein the sheath is fastened to the actuator through a fastener.
Clause 32. The system according to clause 30, wherein the actuator comprises a control unit to control the operation of the motor.
Clause 33. The system according to clause 32, comprising a confirmation switch in electrical or data communication with the control unit to operate the motor.
Clause 34. The system according to clause 33, wherein the confirmation switch is a pedal.
Clause 35. The system according to any of clauses 1-34, wherein the actuator comprises a force sensor to sense a force applied to the wire.
Clause 36. The system according to clause 8, wherein the core comprises a spring-loaded wedge rotatably attached to the movable portion of the core, the wedge being able to move from a retracted status to allow a relative displacement between the movable portion and the fixed portion of the core, to an expanded status biased by a wedge spring to block the relative displacement between the movable portion and the fixed portion.
Clause 37. The system according to any of clauses 1-36, comprising a spring-loaded pawl to engage a number of teeth arranged along the plunger of the syringe in such a way that the plunger is blocked, and a button operatively connected to the pawl in such a way that the pawl is disengaged from the teeth upon actuation of the button.
Clause 38. The system according to clause 37, wherein the button is placed on the cover.
Clause 39. The system according to any of clauses 1-38, wherein the wire is associated with the fixed portion of the core and the sheath is associated with movable portion of the core.
Clause 40. A syringe for the syringe driving system according to clause 37, comprising a number of teeth along a plunger, the teeth being configured to receive the pawl such that a pawl and ratchet mechanism is defined.
Clause 41. An actuator for a hypodermic syringe driving system according to any of clauses 1-39, wherein the actuator has a control unit comprising:
   a receiving module to receive operational data;
   a determining module to determine working parameters of the motor based on the received operational data, wherein the working parameters comprise duration, speed and direction of rotation;
   an activating module to activate the motor based on the determined working parameters.
Clause 42. A method for driving a hypodermic syringe driving system according to any of clauses 1-39, comprising:
   receiving operational data;
   determining working parameters of the motor based on the received operational data, wherein the working parameters comprise at least one of duration, speed and direction of rotation;
   activating the motor based on the determined working parameters.
Clause 43. The method according to clause 42, comprising receiving a confirmation data from the confirmation switch before activating the motor.
Clause 44. The method according to clause 42, wherein the operational data comprise at least one of a volume to be applied, flow rate, and direction of displacement of the movable portion relative to the fixed portion of the core.
Clause 45. The method according to clause 42, wherein the operational data comprise data entered through a user interface.
Clause 46. The method according to clause 42, wherein the operational data comprise data received from a force sensor associated to the wire.
Clause 47. The method according to clause 46, wherein the actuator comprises an alarm unit in electrical communication with the control unit, and the method comprises: activating the alarm unit based on data received from the force sensor.
Clause 48. The method according to clause 41, wherein the motor is activated following a sequence of sets of working parameters.
Clause 49. A method for carrying out a fine needle aspiration biopsy using a hypodermic syringe driving system according to any of clauses 1-39 and a biopsy syringe, comprising:
   receiving operational data;
   determining working parameters of the motor based on the received operational data, wherein the working parameters comprise duration, speed and direction of rotation;
   activating the motor to displace the movable portion with respect to the fixed portion;
   inserting a needle of the syringe into a body of a patient,
   activating the motor to displace the movable portion with respect to the fixed portion, and generating a suction force in the barrel of the syringe;
   obtaining a fluid sample of the patient.
Clause 50. A method for injecting a substance into a patient using a hypodermic syringe driving system according to any of clauses 1-39 and a syringe, comprising:
   receiving operational data;
   determining working parameters of the motor based on the received operational data, wherein the working parameters comprise duration, speed and direction of rotation;
   activating the motor to displace the movable portion with respect to the fixed portion;
   inserting a needle of the syringe into the body of the patient,
   activating the motor to displace the movable portion with respect to the fixed portion, to push the plunger of the syringe;
   expelling a substance through the needle.
Clause 51. A method for injecting a substance and carrying out a fine needle aspiration biopsy using a hypodermic syringe driving system according to any of clauses 1-39 and a syringe, comprising:
   receiving operational data;
   determining working parameters of the motor based on the received operational data, wherein the working parameters comprise duration, speed and direction of rotation;
   activating the motor to displace the movable portion with respect to the fixed portion;
   inserting a needle of the syringe into a body of a patient,
   activating the motor to displace the movable portion with respect to the fixed portion to push the plunger of the syringe to inject an oncolytic virus substance into the body of the patient;
   activating the motor to displace the movable portion with respect to the fixed portion to pull the plunger of the syringe to obtain a fluid sample of the patient.
Clause 52. A hypodermic syringe driving system comprising a remote actuator operatively connected to a handheld body through a Bowden cable, wherein the handheld body comprises:
   a core and a cover to shield the core at least partially, the cover comprises a distal opening to receive a barrel of a syringe and a proximal opening to receive the Bowden cable;
   wherein the core has a movable portion to secure a plunger of the syringe and a fixed portion,
   wherein the Bowden cable has a sheath and a wire, the wire being displaceable with respect to the sheath, the movable and the fixed portion of the core being associated respectively with one of the wire and the sheath in such a way that the movable portion and the fixed portion are displaceable relative to each other along a longitudinal axis of the handheld body.
Clause 53. The system according to clause 52, wherein the cover comprises a retainer slot to receive a retainer, the retainer being attached to the sheath, wherein the retainer comprises a bore to pass the wire through.
Clause 54. The system according to any of clauses 52-53, wherein the core comprises a main spring arranged between the movable portion and the fixed portion such that the movable portion is biased away from the fixed portion.
Clause 55. The system according to any of clauses 52-54, wherein the cover is divided in at least two shells along the longitudinal axis of the handheld body.
Clause 56. The system according to clause 55, wherein the cover comprises a securing mechanism to secure the two shells to each other, the securing mechanism comprising cooperating shapes in such a way that the shapes cooperate to secure the shells when one shell moves relative to the other shell along the longitudinal axis of the handheld body to a closed status of the handheld body.
Clause 57. The system according to any of clauses 52-56, wherein the fixed portion of the core is detachably coupled to the cover.
Clause 58. The system according to any of clauses 52-57, comprising a releasable clip arranged between the fixed portion of the core and the cover such that the fixed portion is releasably secured to an inner wall of the cover.
Clause 59. The system according to any of clauses 52-58, wherein the distal opening comprises a seal to surround the barrel.
Clause 60. The system according to any of clauses 52-59, wherein the fixed portion of the core has a side elongated cut-out to insert the plunger of the syringe.
Clause 61. The system according to any of clauses 52-60, wherein the actuator comprises a motor to drive a pulley to wind and unwind the wire, and the motor and the pulley are connected through a gearbox, wherein the actuator comprises a control unit to control the operation of the motor.
Clause 62. The system according to clause 52, comprising a confirmation switch in electrical or data communication with the control unit to operate the motor.
Clause 63. The system according to any of clauses 61-62, wherein the actuator comprises a force sensor to sense a force applied to the wire.
Clause 64. The system according to clause 54, wherein the core comprises a spring-loaded wedge rotatably attached to the movable portion of the core, the wedge being able to move from a retracted status to allow a relative displacement between the movable portion and the fixed portion of the core, to an expanded status biased by a wedge spring to block the relative displacement between the movable portion and the fixed portion.
Clause 65. The system according to any of clauses 52-64, comprising a spring-loaded pawl to engage a number of teeth arranged along the plunger of the syringe in such a way that the plunger is blocked, and a button operatively connected to the pawl in such a way that the pawl is disengaged from the teeth upon actuation of the button.
Clause 66. An actuator for a hypodermic syringe driving system according to any of clauses 52-55, wherein the actuator has a control unit comprising:
   a receiving module to receive operational data;
   a determining module to determine working parameters of the motor based on the received operational data, wherein the working parameters comprise duration, speed and direction of rotation;
   an activating module to activate the motor based on the determined working parameters.

Although only a number of examples have been disclosed herein, other alternatives, modifications, uses and/or equivalents thereof are possible. Furthermore, all possible combinations of the described examples are also covered. Thus, the scope of the present disclosure should not be limited by particular examples but should be determined only by a fair reading of the claims that follow. If reference signs related to drawings are placed in parentheses in a claim, they are solely for attempting to increase the intelligibility of the claim, and shall not be construed as limiting the scope of the claim.

What is claimed is:

1-47. (canceled)

48. A hypodermic syringe driving system comprising a remote actuator operatively connected to a handheld body through a Bowden cable, wherein the handheld body comprises:
a core and a cover to shield the core at least partially, the cover comprises a distal opening to receive a barrel of a syringe and a proximal opening to receive the Bowden cable; the cover being cylindrically shaped, and the distal opening and the proximal opening being located at opposite ends of the handheld body relative to a longitudinal axis of the handheld body;
wherein the core has a movable portion to secure a plunger of the syringe and a fixed portion,
wherein the fixed portion of the core is detachably coupled to the cover,
wherein the Bowden cable has a sheath and a wire, the wire being displaceable with respect to the sheath, the movable and the fixed portion of the core being associated respectively with one of the wire and the sheath in such a way that the movable portion and the fixed portion are displaceable relative to each other along the longitudinal axis of the handheld body.

49. The system according to claim 48, wherein the cover comprises a retainer slot to receive a retainer, the retainer being attached to the sheath, wherein the retainer comprises a bore to pass the wire through.

50. The system according to claim 49, wherein the retainer comprises a generally flat anchor having a rounded face to be received by the retainer slot.

51. The system according to claim 48, wherein the core comprises a main spring arranged between the movable portion and the fixed portion such that the movable portion is biased away from the fixed portion.

52. The system according to claim 48, wherein the cover is divided in at least two shells along the longitudinal axis of the handheld body.

53. The system according to claim 52, wherein the two shells are detachably joined to each other along the longitudinal axis of the handheld body.

54. The system according to claim 52, wherein each of the shells of the cover comprise respectively a holding pad protruding from an inner face of the shell, the holding pads of each shell being arranged at different points along the longitudinal axis of the handheld body such that a barrel flange of the syringe is sandwiched between the holding pads.

55. The system according to claim 52, wherein the cover comprises a securing mechanism to secure the two shells to each other, the securing mechanism comprising cooperating shapes in such a way that the shapes cooperate to secure the shells when one shell moves relative to the other shell along the longitudinal axis of the handheld body to a closed status of the handheld body, comprising the cooperating shapes a pin arranged in one shell and a hole arranged in the other shell.

56. The system according to claim 48, comprising a releasable clip in such a way that the fixed portion is releasably secured to an inner wall of the cover, wherein the releasable clip has a tab to engage a ledge.

57. The system according to claim 48, wherein the distal opening comprises a seal to surround the barrel.

58. The system according to claim 48, wherein the fixed portion of the core has a side elongated cut-out arranged to insert the plunger of the syringe in an expanded status.

59. The system according to claim 58, wherein the cut-out is disposed in a distal end of the fixed portion.

60. The system according to claim 48, wherein the remote actuator comprises a motor to drive a pulley to wind and unwind the wire, and the motor and the pulley are connected through a gearbox, wherein the actuator comprises a control unit to control the operation of the motor.

61. The system according to claim 60, comprising a confirmation switch in electrical or data communication with the control unit to operate the motor.

62. The system according to claim 48, wherein the actuator comprises a force sensor to sense a force applied to the wire.

63. The system according to claim 51, wherein the core comprises a spring-loaded wedge rotatably attached to the movable portion of the core, the wedge being able to move from a retracted status to allow a relative displacement between the movable portion and the fixed portion of the core, to an expanded status biased by a wedge spring to block the relative displacement between the movable portion and the fixed portion.

64. The system according to claim 48, comprising a spring-loaded pawl to engage a number of teeth arranged along the plunger of the syringe in such a way that the plunger is blocked, and a button operatively connected to the pawl in such a way that the pawl is disengaged from the teeth upon actuation of the button.

65. The system according to claim 48, wherein the wire is associated with the fixed portion of the core and the sheath is associated with movable portion of the core.

66. A method for carrying out a fine needle aspiration biopsy using a hypodermic syringe driving system comprising a remote actuator operatively connected to a handheld body through a Bowden cable, wherein the handheld body comprises:
a core and a cover to shield the core at least partially, the cover comprises a distal opening to receive a barrel of a syringe and a proximal opening to receive the Bowden cable; the cover being cylindrically shaped, and the distal opening and the proximal opening being located at opposite ends of the handheld body relative to a longitudinal axis of the handheld body;
wherein the core has a movable portion to secure a plunger of the syringe and a fixed portion,
wherein the fixed portion of the core is detachably coupled to the cover,
wherein the Bowden cable has a sheath and a wire, the wire being displaceable with respect to the sheath, the movable and the fixed portion of the core being associated respectively with one of the wire and the sheath in such a way that the movable portion and the fixed portion are displaceable relative to each other along the longitudinal axis of the handheld body;
the method comprising:
receiving operational data;
determining working parameters of the motor based on the received operational data, wherein the working parameters comprise duration, speed and direction of rotation;
activating the motor to displace the movable portion with respect to the fixed portion;
inserting a needle of the syringe into a body of a patient,
activating the motor to displace the movable portion with respect to the fixed portion, and generating a suction force in the barrel of the syringe;
obtaining a fluid sample of the patient.

67. A method for injecting a substance and carrying out a fine needle aspiration biopsy using a hypodermic syringe driving system comprising a remote actuator operatively connected to a handheld body through a Bowden cable, wherein the handheld body comprises:
a core and a cover to shield the core at least partially, the cover comprises a distal opening to receive a barrel of a syringe and a proximal opening to receive the Bowden cable; the cover being cylindrically shaped, and the distal opening and the proximal opening being located at opposite ends of the handheld body relative to a longitudinal axis of the handheld body;
wherein the core has a movable portion to secure a plunger of the syringe and a fixed portion,
wherein the fixed portion of the core is detachably coupled to the cover,
wherein the Bowden cable has a sheath and a wire, the wire being displaceable with respect to the sheath, the movable and the fixed portion of the core being associated respectively with one of the wire and the sheath in such a way that the movable portion and the fixed portion are displaceable relative to each other along the longitudinal axis of the handheld body;
the method comprising:
receiving operational data;
determining working parameters of the motor based on the received operational data, wherein the working parameters comprise duration, speed and direction of rotation;
activating the motor to displace the movable portion with respect to the fixed portion;
inserting a needle of the syringe into a body of a patient,
activating the motor to displace the movable portion with respect to the fixed portion to push the plunger of the syringe to inject an oncolytic virus substance into the body of the patient;
activating the motor to displace the movable portion with respect to the fixed portion to pull the plunger of the syringe to obtain a fluid sample of the patient.
